(19) United States
(12) Patent Application Publication
(10) Pub. No.: US 2003/0041345 A1
(43) Pub. Date: Feb. 27, 2003

(54) Receptor-like protein kinases from nicotiana tabacum

(76) Inventors: Thomas Schmulling, Berlin (DE); Silke Schafer, Dusseldorf (DE)

Correspondence Address: BAYER CORPORATION, PATENT DEPARTMENT, 100 BAYER ROAD, PITTSBURGH, PA, 15205, US

(21) Appl. No.: 10/149,846

(22) PCT Filed: Dec. 11, 2000
(86) PCT No.: PCT/EP00/12488

(30) Foreign Application Priority Data
Dec. 20, 1999 (DE) 199 61 519.5

(51) Int. Cl. A01H 1/00; C07H 21/04; C12N 9/12; C12P 21/02; C12N 5/04

(52) U.S. Cl. 800/278; 435/69.1; 435/419; 435/194; 435/320.1; 536/23.2

(57) ABSTRACT

The invention relates to nucleic acids which encode plant polypeptides with the biological activity of receptor-like protein kinases, and to the corresponding polypeptides per se.

The invention relates to nucleic acids which encode plant polypeptides with the biological activity of receptor-like protein kinases, and to the corresponding polypeptides per se.

Receptor-like kinases, as a rule, span the cell membrane and thus have an extracytoplasmic and a cytoplasmic portion. They act in organisms as mediators of signals which are transduced from the outside into the inside of the cell (for example van der Geer at al., 1994). The cytoplasmic protein kinase domain is activated by a ligand binder to the extracellular domain. This is effected by autophosphorylation of either the serine and/or threonine residues, the tyrosine or the histidine residues (for example Fantl et al., 1993). Animal receptor kinases autophosphorylate predominantly tyrosine residues; in contrast, the kinases which are found in plants appear to be virtually exclusively serine/threonine kinases (Becraft, 1998).

Serine/threonine kinases catalyze the reversible transfer of the γ-phophate residue from ATP to amino acids of a receptor protein. The presence of 11 conserved domains determines essentially the enzymatic function of protein kinases (Hanks et al., 1988). A total of 9 amino acids, which are invariable in all of the protein kinases identified to date, are present in these domains. They participate in the binding of ATP and probably the recognition of the amino acid to be phosphorylated, such as serine, threonine or tyrosine.

The present invention relates to nucleic acids which encode plant polypeptides with the biological activity of a receptor-like protein kinase which comprises the amino acid sequence of SEQ ID NO. 2. In particular, the nucleic acids according to the invention encode receptor-like serine/threonine kinases.

The nucleic acids according to the invention are, in particular, single-stranded or double-stranded deoxyribonucleic acids (DNA) or ribonucleic acids (RNA). Preferred embodiments are fragments of genomic DNA which may contain introns, and cDNAs.

The nucleic acids according to the invention are preferably DNA fragments which correspond to genomic DNA from tobacco plants.

The nucleic acids according to the invention especially preferably comprise a sequence selected from among

a) the sequence as shown in SEQ ID NO: 1,

b) sequences which encode a polypeptide which comprises the amino acid sequence as shown in SEQ ID NO: 2,

c) part-sequences of the sequences defined under a) or b) which are at least 14 base pairs in length,

d) sequences which hybridize with the sequences defined under a) or b),

e) sequences which have at least 60% identity, preferably at least 80% identity, especially preferably at least 90% identity with the sequences defined under a) or b),

f) sequences which have at least 60% identity, preferably at least 80% identity, especially preferably at least 90% identity with the N-terminal receptor domain of the sequences defined under a) or b),

g) sequences which are complementary to the sequences defined under a) or b), and

h) sequences which, owing to the degeneracy of the genetic code, encode the same amino acid sequence as the sequences defined under a) to f).

A cDNA molecule with the sequence as shown in SEQ ID NO: 1 represents a very especially preferred embodiment of the nucleic acids according to the invention.

The term “to hybridize” as used in the present context describes the process in which a single-stranded nucleic acid molecule undergoes base pairing with a complementary strand. Starting from the sequence information disclosed herein, it is possible in this manner, for example, to isolate DNA fragments which encode receptor-like protein kinases with the same or similar properties as the kinase with the amino acid sequence as shown in SEQ ID NO: 2 from plants other than tobacco plants.

Hybridization conditions are calculated by approximation using the following formula:

Melting temperature Tm=81.5° C.+16.6 log{c(Na⁺)]+0.41(% G+C))−500/n (Lottspeich and Zorbas, 1998).

In this formula, c is the concentration and n the length of the hybridizing sequence segment in base pairs. For a sequence >100 bp, the term 500/n is dropped. At the highest stringency, washing is effected at a temperature of 5-15° C. below Tm and an ionic strength of 15 mM Na⁺ (corresponds to 0.1×SSC). If an RNA sample is used for hybridization, the melting point is 10-15° C. higher.

Preferred hybridization conditions are stated hereinbelow:

Hybridization solution: 6×SSC/5X Denhardt's solution/50% formamide;

Hybridization temperature: 36° C., preferably 42° C.;

Wash step 1: 2×SSC, 30 minutes, at room temperature;

Wash step 2: 1×SSC, 30 minutes at 50° C.; preferably 0.5×SSC, 30 minutes at 50° C.; especially preferably 0.2×SSC, 30 minutes at 65° C.

The term “N-terminal receptor domain” as used in the present context refers to a peptide region which corresponds functionally to the peptide region with an amino acid sequence from position 1 through position 394 of the sequence as shown in SEQ ID NO: 2.

The degree of identity of the nucleic acids is preferably determined with the aid of the program NCBI BLASTN Version 2.0.4. (Altschul et al., 1997).

The present invention also relates to the regulatory regions which naturally control the transcription of the nucleic acids according to the invention in plant cells, especially in tobacco plants.

The term “regulatory regions” as used in the present context relates to untranslated regions of the gene in question, such as promoters, enhancers, binding sites for repressors or activators, or termination sequences, which interact with cellular proteins, thus controlling the transcription.

The present invention furthermore relates to DNA constructs comprising a nucleic acid according to the invention and a heterologous promoter.

The term “heterologous promoter” as used in the present context refers to a promoter which has properties other than the promoter which controls the expression of the gene in question in the original organism.

The choice of heterologous promoters depends on whether pro- or eukaryotic cells or cell-free systems are used for expression. Examples of heterologous promoters are the cauliflower mosaic virus 35S promoter for plant cells, the alcohol dehydrogenase promoter for yeast cells, the T3, T7 or SP6 promoters for prokaryotic cells or cell-free systems.

The present invention furthermore relates to vectors comprising a nucleic acid according to the invention, a regulatory region according to the invention or a DNA construct according to the invention. All of the phages, plasmids, phagmids, phasmids, cosmids, YACs, BACs, artificial chromosomes or particles suitable for particle bombardment which are used in molecular biology laboratories may be employed as vectors.

Preferred vectors are pBIN (Bevan, 1984) and its derivatives for plant cells, pFL61 (Minet et al., 1992) for yeast cells, pBLUESCRIPT vectors for bacterial cells, lamdaZAP (Stratagene) for phages.

The present invention also relates to host cells comprising a nucleic acid according to the invention, a DNA construct according to the invention or a vector according to the invention.

The term “host cells” as used in the present context refers to cells which do not naturally comprise the nucleic acids according to the invention.

Suitable host cells are prokaryotic cells, preferably E. coli, or else eukaryotic cells such as cells of Saccharomyces cerevisiae, Pichia pastoris, insects, plants, froschoocytes and mammalian cell lines.

The present invention furthermore relates to polypeptides with the biological activity of receptor-like protein kinases which are encoded by the nucleic acid according to the invention. In particular, these are polypeptides which represent serine/threonine kinases according to the invention.

The term “polypeptides” as used in the present context not only relates to short amino acid chains which are usually termed peptides, oligopeptides or oligomers, but also to longer amino acid chains which are usually termed proteins. It comprises amino acid chains which can be modified either by natural processes, such as posttranslational processing, or by chemical prior-art methods. Such modifications may occur at various sites and repeatedly in a polypeptide, such as, for example, on the peptide backbone, on the amino acid side chain, on the amino and/or the carboxyl terminus. For example, they encompass acetylations, acylations, ADP ribosylations, amidations, covalent linkages with flavins, with hem moities, with nucleotides or nucleotide derivatives, with lipids or lipid derivatives or with phosphatidylinositol, cyclizations, disulfide bridge formations, demethylations, cystin formation, formylations, gamma-carboxylations, glycosylations, hydroxylations, iodinations, methylations, myristoylations, oxidations, proteolytic processings, phosphorylations, selenoylations and tRNA-mediated amino acid additions.

The polypeptides according to the invention may exist in the form of “mature” proteins or parts of larger proteins, for example as fusion proteins. They can furthermore exhibit secretion or leader sequences, pro-sequences, sequences which allow simple purification, such as polyhistidine residues, or additional stabilizing amino acids.

The polypeptides according to the invention need not constitute complete receptors, but may also be fragments thereof, as long as they maintain at least one biological activity of the complete protein kinases. Polypeptides with the same biological activity as a receptor-like protein kinase with an amino acid sequence as shown in SEQ ID NO: 2 are still considered to be in accordance with the invention. In this context, the polypeptides according to the invention need not be deducible from receptor-like protein kinases from tobacco. Polypeptides which are also considered as being in accordance with the invention are those which correspond to receptor-like protein kinases of, for example the following plants, or to fragments of these receptor-like protein kinases which are still capable of exerting their biological activity: maize, wheat, barley, oats, rice, rye, tomatoes, legumes, potato plants, Lactuca sativa, Brassicaceae, woody species, Physcomitrella patens.

In comparison with the corresponding region naturally occurring in receptor-like protein kinases, the polypeptides according to the invention can have deletions or amino acid substitutions, as long as they retain at least one biological activity of the complete receptors. Conservative substitutions are preferred. Such conservative substitutions encompass variations, one amino acid being replaced by another amino acid from among the following group:

1. Small aliphatic residues, unpolar residues or residues of little polarity: Ala, Ser, Thr, Pro and Gly;

2. Polar, negatively charged residues and their amides: Asp, Asn, Glu and Gln;

3. Polar, positively charged residues: His, Arg and Lys;

4. Large aliphatic unpolar residues: Met, Leu, Ile, Val and Cys; and

5. Aromatic residues: Phe, Tyr and Trp.

Preferred conservative substitutions can be seen from the following list:

| Original residue | Substitution |
|---|---|
| Ala | Gly, Ser |
| Arg | Lys |
| Asn | Gln, His |
| Asp | Glu |
| Cys | Ser |
| Gln | Asn |
| Glu | Asp |
| Gly | Ala, Pro |
| His | Asn, Gln |
| Ile | Leu, Val |
| Leu | Ile, Val |
| Lys | Arg, Gln, Glu |
| Met | Leu, Tyr, Ile |
| Phe | Met, Leu, Tyr |
| Ser | Thr |
| Thr | Ser |
| Trp | Tyr |
| Tyr | Trp, Phe |
| Val | Ile, Leu |

The present invention therefore also relates to polypeptides which exert at least one biological activity of a receptor-like protein kinase and which comprises an amino acid sequence which has at least 60% identity, preferably at least 80% identity, especially preferably at least 90% identity, very especially preferably 97-99% identity with the sequence as shown in SEQ ID NO: 2 over its entire length.

The degree of identity of the amino acid sequences is preferably determined with the aid of the program BLASTP+BEAUTY Version 2.0 4. (Altschul et al., 1997).

A preferred embodiment of the polypeptides according to the invention is the cyto-kinin-regulated receptor-like protein kinase (CRK1) with the amino acid sequence as shown in SEQ ID NO: 2.

In the CRK1 amino acid sequence, the 11 kinase domains in which all of the amino acids defined by Hanks et al., 1988, are present. It is therefore a serine/threonine kinase. The CRK1 protein has a transmembrane domain in the region of amino acids 390 and 410. The first 28 amino acids with a positively charged amino acid residue and a hydrophobic region of 15 amino acids of the N terminus constitute a signal sequence. The cleavage site is probably located between amino acids 28 and 29. This signal sequence ensures translocation of the newly synthesized CRK1 protein across the membrane of the endoplasmic reticulum and further transport into the plasma membrane. The N-terminal domain of CRK1 between amino acids 29 and 390 is located extracytoplasmically. This extracellular domain encompasses four regions which have >70% homology with ATP/GTP binding sites or binding sites for cyclic nucleotides. In the case of CRK1, cytokinins are possible ligands. As adenine derivatives, cytokinins are structurally similar to purines.

Cytokinins belong to the group of the plant hormones and have the ability of inducing cell division. So far, over 40 naturally occurring cytokinins have been isolated, and all of the known cytokinins are adenine derivatives. The activity is determined by the type of the substituents, above all the N⁶ group (reviewed by Shaw, 1994 and Kaminek, 1992).

The spectrum of actions which have been associated with cytokinins is varied. Besides the induction of cell division in combination with auxin, cytokinins also affect differentiation processes. A high cytokinin:auxin ratio brings about shoot formation in callus in vitro, while a high auxin:cytokinin ratio brings about root formation (Skoog and Miller, 1957). Cytokinins cause reduced apical dominance, which results in the lateral buds breaking (Wickson and Thimann, 1958). Cytokinins also play an important role in delaying leaf senescence. In this context, cytokinins cause, inter alia, an inactivation of proteolytic enzymes, of lipases and lipoxy kinases, which are responsible for the gradation processes. Further traditional cytokinin effects include the promoting influence on chloroplast development and the inhibition of longitudinal root growth.

Cytokinins are necessary for normal plant development. An undue supply of this hormone leads to extremely compact sterile plants.

Since the amount of CRK1 transcripts is regulated by cytokinins, it can constitute a component of the cytokinin signal transduction pathway. The amount of CRK1 transcript is regulated by cytokinins in a specific, transient manner. The CRK1 gene is subject to early regulation; 30 minutes after addition of 5×10⁻⁷ M BAP, the amount of transcript is virtually fully reduced. Depending on the cytokinin concentration, the CRK1 transcripts accumulate again after 9-24 hours of cytokinin treatment, so that transient regulation may be assumed. This regulation might constitute a negative feed-back mechanism in order to ensure that, while a cell is competent for a signal, this sensitivity may be altered by a reduced protein quantity of signal transduction components. It is postulated for the CRK1 receptor that, once the cell has received the signal, it is not capable for a certain period of time of responding to further cytokinin signals.

When determining the dose dependency of the regulation, a very low cytokinin concentration of 5×10⁻¹² M BAP proves to be sufficient for fully reducing the amount of transcript.

Other plant hormones, such as gibberellic acid, ACC, brassinosteroids and jasmonic acid, and the structural analog adenine, do not result in a modification of the abundance of CRK1 transcripts when applied in comparable concentrations. Auxin and abscisic acid can result in a decreased abundance of CRK1 transcript when applied in considerably higher concentrations than cytokinin.

The term “biological activity of a receptor-like protein kinase” as in the present context denotes a modification of cell activity and plant growth following ligand binding.

The present invention furthermore relates to antibodies which specifically bind to the polypeptides according to the invention. Such antibodies are raised in the customary manner. For example, these antibodies may be used to identify expression clones, for example of a gene library, which carry the nucleic acids according to the invention.

The term “antibody” as used in the present context also extends to parts of complete antibodies, such as Fa, F(ab′)₂ or Fv fragments, which retain the ability of binding to epitopes of the polypeptides according to the invention.

The present invention furthermore also relates to processes for the preparation of the nucleic acids according to the invention. The nucleic acids according to the invention can be prepared in the customary manner. For example, the nucleic acid molecules can be prepared exclusively by chemical synthesis. Alternatively, short segments of the nucleic acids according to the invention can be synthesized chemically and such oligonucleotides can be radiolabeled or labeled with a fluorescent dye. The labeled oligonucleotides may also be used to screen cDNA libraries prepared starting from plant mRNA. Clones which hybridize with the labeled oliogonucleotides are selected to isolate the DNA fragments in question. After the DNA isolated has been characterized, the nucleic acids according to the invention are obtained in a simple manner.

The nucleic acids according to the invention may also be prepared by means of PCR methods using chemically synthesized oligonucleotides.

The term “oligonucleotide(s)” as used in the present context denotes DNA molecules composed of 10 to 50 nucleotides, preferably 15 to 30 nucleotides. They are synthesized chemically and can be used as probes.

The present invention furthermore relates to processes for the preparation of polypeptides according to the invention. To prepare the polypeptides which are encoded by the nucleic acids according to the invention, host cells comprising the nucleic acids according to the invention can be cultured under suitable conditions. Then, the desired polypeptides can be isolated from the cells or the culture medium in the customary manner. The polypeptides can also be prepared in in-vitro systems.

A rapid method of isolating the polypeptides according to the invention which are synthesized by host cells using a nucleic acid according to the invention starts with expressing a fusion protein, it being possible for the fusion moiety to be affinity-purified in a simple manner. The fusion moiety can be, for example, glutathione S-transferase. In this case, the fusion protein can be purified on a glutathione affinity column. The fusion moiety can be removed by partial proteolytic cleavage for example at linkers between the fusion moiety and the polypeptide according to the invention which is to be purified. The linker can be designed in such a way that it includes target amino acids, such as arginine and lysine residues, which define sites for trypsin cleavage. Standard cloning methods using oligonucleotides may be employed to prepare such linkers.

Other purification processes which are possible are based on preparative electrophoresis, FPLC, HPLC (for example using gel filtration columns, reversed-phase columns or mildly hydrophobic columns), gel filtration, differential precipitation, ion-exchange chromatography and affinity chromatography.

Since receptor-like protein kinases constitute membrane proteins, the purification methods preferably rely on detergent extractions, for example using detergents which have no or only little effect on the secondary and tertiary structures of the polypeptides such as nonionic detergents.

The purification of the polypeptides according to the invention can comprise the isolation of membranes starting from host cells which express the nucleic acids according to the invention. Preferably, such cells express a sufficient copy number of the polypeptides according to the invention, so that the amount of the polypeptides in a membrane fraction is at least 10 times higher than the amount found in comparable membranes of cells which naturally express the CRK1 gene; especially preferably, the amount is at least 100 times higher, very especially preferably at least 1000 times higher.

The term “isolation or purification” as used in the present context means that the polypeptides according to the invention are separated from other proteins or other macromolecules of the cell or of the tissue. Preferably, a composition comprising the polypeptides according to the invention is at least 10-fold concentrated, especially preferably at least 100-fold concentrated, with regard to the protein content in comparison with the preparation from the host cells.

The polypeptides according to the invention can also be affinity-purified without fusion moiety with the aid of antibodies which bind to the polypeptides.

The present invention also relates to processes of finding chemical compounds which bind to the polypeptides according to the invention and modify their properties. Owing to the wide range of functions of the receptor-like protein kinases according to the invention, modulators which affect the activity may constitute novel growth-regulatory or herbicidal active compounds.

The term “agonist” as used in the present context refers to a molecule which accelerates or enhances the signal transduction of the receptor-like protein kinases according to the invention, i.e. the autophosphorylation of the serine and/or threonine residues.

The term “antagonist” as used in the present context refers to a molecule which slows down or prevents the signal transduction of the receptor-like protein kinases according to the invention, i.e. the autophosphorylation of the serine and/or threonine residues.

The term “modulator” as used in the present context constitutes the generic term for agonist and antagonist. Modulators can be small organochemical molecules, peptides or antibodies which bind to the polypeptides according to the invention. Modulators may furthermore be small organochemical molecules, peptides or antibodies which bind to a molecule which, in turn, binds to the polypeptides according to the invention, thus affecting their biological activity. Modulators may constitute natural substrates and ligands or their structural or functional mimetics. However, the term “modulator” does not encompass cytokinins.

The modulators are preferably small organochemical compounds.

The binding of the modulators to the polypeptides according to the invention can alter the cellular processes in a manner which leads to the death of the plants treated therewith.

The present invention furthermore contemplates processes of finding chemical compounds which modify the expression of the polypeptides according to the invention. Such “expression modulators” too may constitute novel growth-regulatory or herbicidal active compounds. Expression modulators can be small organochemical molecules, peptides or antibodies which bind to the regulatory regions of the nucleic acids encoding the polypeptides according to the invention. Expression modulators may furthermore be small organochemical molecules, peptides or antibodies which bind to a molecule which, in turn, binds to regulatory regions of the nucleic acids encoding the polypeptides according to the invention, thus affecting their expression. Expression modulators may also be antisense molecules.

The present invention therefore also extends to the use of modulators of the polypeptides according to the invention or of expression modulators as plant growth regulators or herbicides.

The processes according to the invention include high throughput screening (HTS). Not only host cells, but also cell-free preparations comprising the nucleic acids according to the invention and/or polypeptides according to the invention may be used for this purpose.

To find modulators, a synthetic reaction mix (for example in-vitro transcription products) or a cellular component, such as a membrane or any other preparation comprising the polypeptides according to the invention, may be incubated together with a labeled substrate or ligand of the polypeptides in the presence and absence of a candidate molecule which may be an agonist or antagonist. The capability of the candidate molecule of increasing or inhibiting the activity of the polypeptides according to the invention can be seen from an increased or reduced binding of the labeled ligand or an increased or reduced conversion of the labeled substrate. Molecules which bind well and lead to an increased activity of the polypeptides according to the invention are agonists. Agonists which bind well but do not trigger the biological activity of the polypeptides according to the invention are probably good antagonists. The detection of the biological activity of the polypeptides according to the invention can be improved by what is known as a reporter system. Reporter systems in this context comprise, but are not limited to, colorimetrically labeled substrates which are converted into a product, or a reporter gene which responds to changes in the activity or the expression of the polypeptides according to the invention, or other known binding tests.

Another example of a method by means of which modulators of the polypeptides according to the invention can be found is a displacement test in which the polypeptides according to the invention and a potential modulator are combined under conditions suitable for this purpose with a molecule which is known to bind to the polypeptides according to the invention, such as a natural substrate or ligand or a substrate or ligand mimetic. The polypeptides according to the invention themselves can be labeled, for example radiolabeled or calorimetrically labeled, so that the number of the polypeptides which are bound to a ligand or which have undergone conversion can be determined accurately. In this manner, the efficacy of an agonist or antagonist can be determined.

The invention furthermore relates to the use of a nucleic acid according to the invention, to a DNA construct according to the invention or to a vector according to the invention for the generation of transgenic plants, and to the corresponding transgenic plants as such or their parts or propagation material.

Transgenic plants, plant parts, protoplasts, plant tissues or plant propagation materials in which the intracellular concentration of the receptor-like protein kinases is increased or reduced in comparison with the corresponding wild-type forms after introduction of a nucleic acid according to the invention, a DNA construct according to the invention or a vector according to the invention are also subject matter of the present invention.

The term “plant parts” as used in the present context denotes all aerial and subterraneous parts and organs of the plants, such as shoot, leaf, flower and root, and protoplasts and tissue cultures prepared with them.

The term “propagation material” as used in the present context denotes vegetative and generative propagation material, such as cuttings, tubers, rhizomes, shoots and seeds.

The invention also relates to plants, plant parts, protoplasts, plant tissues or plant propagation materials in which modifications in the sequence encoding endogenous receptor kinases have been carried out and selected which lead to the preparation of a receptor kinase according to the invention, or in which an increase or reduction of the endogenous receptor kinase activity is obtained by mutagenesis.

For example, endogenous receptor kinase genes which are already present in the plant can be modified by mutagenesis, for example with ethyl methanosulfonate (EMS). After mutagenesis, individuals which, owing to the mutagenesis, produce receptor kinases according to the invention with an increased or reduced activity can be selected specifically by sequence analysis, ligand-binding studies or analysis of a biochemical reaction brought about by ligand binding. In the same manner, the expression of a receptor kinase gene according to the invention already present in the plant can be modified by mutagenesis of the regulatory sequences in such a way that plants with a reduced or increased ligand sensitivity are obtained. Such plants can be identified by generally known methods of gene expression analysis, such as Northern blot or Western blot.

Since the receptor-like protein kinases according to the invention, in particular the CRK1 receptor, plays a role in signal perception, an increased ligand sensitivity is obtained in transgenic “sense” plants or in plants which were selected specifically for an increased amount or activity of corresponding endogenous receptor kinases or elements linked thereto in signal transduction. Firstly, lesser ligand concentrations may already be detected, and transduced as a signal, owing to such modifications, or else a ligand effect may be obtained in the absence of a ligand by switching on the signal transduction pathway; secondly, no negative feedback may occur upon constitute expression so that the cells are permanently competent for the signal. A response to this situation is increased ligand effects. Transgenic “antisense” plants or plants with a reduced receptor kinase activity have a reduced ligand sensitivity. An increase or reduction in the receptor kinase activity can give rise to plants with a modified development, modified physiology or modified morphology.

### EXAMPLES

### Example 1

Isolation of the Above-described Nucleotide Sequence CRK1:

A 400 bp CRK1 fragment was isolated from cell suspension cultures of Nicotiana tabacum L. cultivar Wisconsin 38 (Skoog and Miller, 1957) with the aid of the representational difference analysis (RDA) method (Hubank and Schatz, 1994).

Representational difference analysis (RDA) is a method for isolating differentially expressed genes. The two samples to be compared were a tobacco cell culture which had grown without added cytokinin and a cell culture to which 10⁻⁷ M BAP (benzyl-aminopurine) had been added for 45 minutes after 5 days of subculturing.

The fragments obtained were cloned into vector pUC19. The CRK1 fragment was also used as probe for Northern and Southern blots and employed as probe in a cDNA library screening to isolate the complete CRK1 cDNA.

Isolation of the Complete cDNA Sequence of CRK1 (Modified Stratagene Method)

A cDNA library was generated by SCInet in the λ-ZAP-Express vector following the instructions of the manufacturer Stratagene. To this end SCInet was provided with 400 μg of total RNA of the tobacco suspensions culture W38. Previously, it had been ensured that the desired mRNA is expressed in the culture. After the finished cDNA library had been obtained, the titer was checked, and the complete CRK1-cDNA was searched for in a five-step screen. The titer of the cDNA library was 1×10⁹ pfu/ml.

200 μl E. coli XL1-Blue MRF' cells were incubated for 20 minutes at RT with 100,000 pfu of the cDNA library in question, 7 ml of top agar were added, and the mixture was poured into prewarmed (37° C.) NZY agar plates (diameter 145 mm). After incubation overnight at 37° C., phages with an insertion which is homologous to the probe used were identified.

Primary Screen:

The phage plates were cooled for 1 hour at 4° C., nylon filters were placed on them for 1 minute, and, with the DNA side facing up, incubated for 2 minutes on Whatman 3M filter paper fixed with denaturing solution and for 5 minutes on Whatman 3M filter paper soaked with neutralization solution. The filter was briefly immersed in a 2×SSC solution, dried and then irradiated using UV light to immobilize the DNA (UV Transilluminator, 0.12 J/cm²).

After positive plaques had been identified, they were excised generously (approx. 1 cm²) in the primary screening, using a scalpel, and shaken for at least one hour together with 3-5 ml of SM buffer. 1 μl of the suspension, and 1 μl of a 1:10 dilution, were used for a secondary screening. Positive plaques in the secondary screening were punched out with sterile Pasteur pipettes, dissolved in 500 μl of SM buffer, and 30 μl of a 10⁻² dilution were used for a tertiary screening. For a further screening cycle, the same dilution was used, and individual plaques were obtained in some cases. These positive individual plaques were used to carry out a fifth cycle, in which all of the individual plaques obtained on the plate had to be positive in order to process the clone in question there. The cDNA inserts of these individual plaques were converted into plasmid DNA by an excision reaction in an E. coli strain XLOLR and isolated. The procedure was as described by the manufacturer Stratagene.

5′/3′-Race

The 5′/3′-RACE method was employed to isolate 5′ and 3′ ends, which had been missing in the cDNA clone identified.

The 5′-RACE is based on the specific amplification of the 5′ end of a gene from mRNA. The cDNA first strand is synthesized with the aid of a sequence-specific primer and AMV reverse transcriptase. A poly(A) tail is attached to the product, so that an oligo dT anchor primer and a nested sequence-specific primer can be employed in the subsequent PCR. A further nested primer may be used in a second PCR in order to ensure specificity.

For the 3′-RACE, the cDNA first-strand synthesis is effected with an oligo dT primer and the subsequent PCR reactions are effected with sequence-specific primers. The products were cloned using the “TA cloning kit” from Pharmacia.

### Example 2

Northern blot analyses were carried out to verify a differential expression of CRK1 in response to cytokinin.

Isolation of Total RNA from Tobacco Suspension Cultures (Puissant and Houdebine, 1990)

To obtain total RNA from suspension cell cultures, the cells were dried briefly on a suction filter which had been connected to a vacuum pump. The material was frozen in liquid nitrogen and stored at −80° C. until the beginning of the RNA isolation.

1 g of plant material is homogenized with a pestle and mortar at 4° C. together with 5 ml of cooled RNA isolation buffer. The homogenized plant material is transferred into Greiner tubes. 0.5 ml of 2 M sodium acetate (pH 4.0) is added and the mixture is vortexed. After 5 ml of phenol has been added, the solution is vortexed again. To improve phase separation, 1 ml of chloroform is added, and the mixture is centrifuged for 10 minutes at 4000 rpm. The aqueous phase is drawn off and transferred into fresh Greiner tubes. The RNA is precipitated with 0.7 parts by volume of isopropanol and centrifugation for 10 minutes at 4000 rpm. The collected pellet is resuspended in 2 ml of 4 M lithium chloride and again centrifuged for 10 minutes at 4000 rpm to remove polysaccharides. The pellet is resuspended in 2 ml of TES buffer and, to remove proteins and phenol residues, mixed with 2 ml of chloroform and centrifuged for 10 minutes at 4000 rpm to separate the phases. The aqueous phase is drawn off, transferred into Eppendorf tubes, and the RNA is precipitated with 0.7 part by volume of isopropanol and an end concentration of 0.3 M sodium acetate (pH 5.2). The mixture is centrifuged for 30 minutes at 4000 rpm. Salts are then eluted from the RNA pellet using 70% alcohol. The mixture is centrifuged for 10 minutes at 4000 rpm. After the alcohol has been drawn off, the pellet is dried in vacuo and dissolved in 17 μl of TE.

Isolation of PolyA-mRNA from Total RNA Preparations (Following the Protocol of the Manufacturer: Dynal)

The purification principle is based on the removal of polyadenylated mRNA by oligo-dT-coated Dynabeads following the manufacturer's instructions. The mRNA yield was approximately 1-2% of the original amount of total RNA employed.

The procedure followed the manufacturer's instructions.

The agarose is made up and boiled up in distilled H₂O with an end concentration of 1.5% agarose. After the mixture has cooled to 60° C., 1/10 parts by volume of 10×MOPS buffer and 1/6 parts by volume of formaldehyde (37%) are added. To check identically applied quantities, EtBr is added. After solidification, the gel is set for 10 minutes at 19 V. 50 μg of the nucleic acid samples and 3 μl of the sized standard (RNA: 0.24-9.5 kb RNA Ladder from Gibco BRL Lifetechnologies) are treated with 2 parts by volume of sample buffer and denatured for 10 minutes at 65° C. in a heating block. The RNA samples are separated by electrophoresis for approximately 5 hours at 45 V.

Transfer of the RNA Through a Nylon Membrane

The nucleic acid samples which have been separated by electrophoresis in a denaturing agarose formaldehyde gel are blotted onto nylon membrane. To this end, 3M Whatman blotting paper is soaked in 10×SSC buffer on a glass sheet and arranged without air bubbles over two containers in such a way that its ends project into the containers, which are also filled with 10×SSC buffer. Avoiding air bubbles, the gel is placed upside down on the 3M Whatman blotting paper and covered with a nylon membrane. The size of the membrane should not exceed the size of the gel and the membrane should lie on the gel without air bubbles. Then, two layers of 3M Whatman blotting paper are placed on the nylon membrane. The agarose gel is now sealed with film in such a way that no liquid which is drawn up can bypass it.

Approximately 5 cm of blotting paper, a larger glass sheet and, thereon, a weight of approximately half a kilogram are placed on the 3M Whatman blotting paper. Blotting is effected for at least 6 hours. The RNA is immobilized by UV crosslinking with a Fluo Link UV lamp. The dose is 0.12 J/cm².

Radiolabeling by In-vitro Transcription

The fragment to be labeled must be cloned into a vector which has a T3 and/or a T7 promoter, for example pBluescript. The plasmid is digested with a restriction enzyme which is positioned downstream of the sequence to be transcribed in order to prevent transcription of all of the plasmid. After digestion with a proteinase K, the DNA is extracted by shaking with phenol/chloroform and precipitated.

The sequence of 1.5 clone was excised from the pUC19 vector via Eco RI and Xba I cleavage sites and cloned into the pBluescript SK vector via the same restriction cleavage site. An antisense RNA can be synthesized with T3 polymerase using the T3 promoter. 1 μg of DNA was employed for labeling. The procedure was carried out with the “in vitro transcription” kit from Stratagene and the manufacturer's instructions were followed. Following the “in vitro transcription”, a Dnase digest was carried out for 15 minutes. The RNA was extracted by shaking and precipitated phenol/chloroform. The incorporation rate averaged 5×10⁷ cpm/μg DNA. The labeled probe was denatured and added to the prehybridized membrane.

Removal of Unincorporated Nucleotides

A Sephadex G50 column (“Nick Columns”, Pharmacia) is washed with 10 ml of water. The labeling mix and 350 μl of water are applied with a pipette, and the eluate is discarded. Another 400 μl of water are applied to the column. The eluate contains the labeled sample. 1 μl of the eluate are removed to determine the specific activity (cpm/μg DNA), which should amount to at least 10⁸ cpm/μg of DNA employed. The remainder of the eluate is denatured for 10 minutes at 95° C., cooled briefly on ice, and placed into the hybridization tube.

Hybridization with ³²P-labeled Nucleic Acid Fragments

The nylon membrane with immobilized nucleic acid is placed into a hybridization tube. To block unspecific binding sites on the membrane, a prehybridization with herring sperm is first carried out. To this end, 10 ml of hybridization solution per 10 cm² of membrane are placed into the hybridization tube, and 1 ml of freshly denaturated herring sperm solution (10 μg/ml) are added per 10 ml of hybridization solution. Prehybridization, and hybridization, are carried out in a hybridization oven at 68° C. Prehybridization should take at least half an hour, and the labeled and freshly denatured DNA sample is then added. Hybridization is carried out overnight.

The next day, unhybridized DNA sample and unspecific binding are washed from the membrane. To this end, the membrane is first washed twice with 2×SSC, 0.1% SDS solution for in each case 15 minutes at room temperature and subsequently for 30 minutes at hybridization temperature with 0.2×SSC, 0.1% SDS solution.

The membrane is sealed into a film, and an X-ray film is placed thereon for detection.

References

Altschul, S. F., Madden, T. L., Schaffer, A. A., Zhang, J. Z.; Miller W. and Lipman, D. J. 1997. Gapped BLAST and PSI-BLAST generation of protein database search programs. Nucleic Acids Res. 25: 3389-3402.

Becraft, P. W. 1998. Receptor kinases in plant development. TIPS 3: 384-388.

Bevan M. 1984. Binary Agrobacterium vectors for plant transformation. Nucleic Acids Res 12(22): 8711-8721.

Fantl, W. J., Johnson, D. E. and Williams, L. T. 1993. Signalling by receptor tyrosine kinases Ann. Rev. Biochem. 62: 453-481.

Hanks, S. K., Quinn, A. M. and Hunter, T. 1988. The protein kinase family: Conserved features and deduced phylogeny of the catalytic domains. Science 241: 42-52.

Hubank, M. and Schatz, D. G. 1994. Identifying differences in mRNA expression by representational difference analysis of cDNA. Nucl. Acids Res. 22: 5640-5648.

Kaminek, M. 1992. Progress in cytokinin research. TIBTECH 10, 159-164.

Lottspeich, F., Zorbas H. (Ed.). 1998. Bioanalytik. Spektrum Akademischer Verlag, Heidelberg, Berlin.

Minet, M., Dufour, M. -E. and Lacroute, F. 1992. Complementation of Saccharomyces cerevisiae auxotrophic mutants by Arabidopsis thaliana cDNAs. Plant J. 2: 417-422.

Puissant, C. and Houdebine, L. -M. 1990. An improvement of the single-step method of the RNA isolation by acid guanidinium thiocyanate-phenol-chloroform extraction. BioTechniques 8: 148-149.

Shaw, G. 1994. Chemistry of adenine cytokinins. In: Mok, D. and Mok, M. (Ed.). Cytokinins, CRC Press, Boca Raton: 15-34.

Skoog, F., Miller, C. O. 1957. Chemical regeneration of growth and organ formation in plant tissue cultures in vitro. Symp. Soc. Exp. Biol. 11: 118-131.

Van der Geer, P. Hunter, T. and Lindberg, R. A. 1994. Receptor protein-tyrosine kinases and their signal transduction pathways. Ann. Rev. Cell Biol. 10: 251-337.

Wickson, M. und Thimann, K. V. 1958. The antagonism of auxin and kinetin in apical dominance. Physiol. Plant. 11: 62.

1. Nucleic acids which encode plant polypeptides with the biological activity of a receptor-like protein kinase which comprises the amino acid sequence as shown in SEQ ID NO: 2.

2. Nucleic acids according to claim 1, characterized in that they encode polypeptides with the activity of serine/threonine kinases.

3. Nucleic acids according to claim 1 or 2, characterized in that they are single-stranded or double-stranded DNA or RNA.

4. Nucleic acids according to claim 3, characterized in that they are fragments of genomic DNA or cDNA.

5. Nucleic acids according to any of claims 1 to 4, characterized in that they are derived from tobacco plants.

6. Nucleic acids according to any of claims 1 to 5, comprising a sequence selected from among
(a) the sequence as shown in SEQ ID NO: 1,
(b) sequences which encode a polypeptide which comprises the amino acid sequence as shown in SEQ ID NO: 2,
(c) part-sequences of the sequences defined under a) or b) which are at least 14 base pairs in length,
(d) sequences which hybridize with the sequences defined under a) or b),
(e) sequences which have at least 60% identity with the sequences defined under a) or b),
(f) sequences which have at least 60% identity with the N-terminal receptor domain of the sequences defined under a) or b),
(g) sequences which are complementary to the sequences defined under a) or b), and
(h) sequences which, owing to the degeneracy of the genetic code, encode the same amino acid sequence as the sequences defined under a) to e).

7. Regulatory region which naturally controls the transcription of a nucleic acid according to any of claims 1 to 6 in plant cells, in particular in tobacco plants.

8. DNA construct comprising a nucleic acid according to any of claims 1 to 6 and a heterologous promoter.

9. Vector comprising a nucleic acid according to any of claims 1 to 6, a regulatory region according to claim 7 or a DNA construct according to claim 8.

10. Vector according to claim 9, characterized in that the nucleic acid is linked operably to regulatory sequences which ensure the expression of the nucleic acid in pro- or eukaryotic cells.

11. Host cell comprising a nucleic acid according to any of claims 1 to 6, a DNA construct according to claim 8 or a vector according to claim 9 or 10.

12. Host cell according to claim 11, characterized in that it is a prokaryotic cell, in particular E.coli.

13. Host cell according to claim 11, characterized in that it is a eukaryotic cell, in particular a yeast, insect, mammalian or plant cell.

14. Polypeptide with the biological activity of a receptor-like kinase, which polypeptide is encoded by a nucleic acid according to any of claims 1 to 6.

15. Polypeptide with the biological activity of a receptor-like kinase, which polypeptide comprises an amino acid sequence with at least 60% identity with the sequence as shown in SEQ ID NO: 2.

16. Antibody which binds specifically to a polypeptide according to claim 14 or 15.

17. Process of preparing a nucleic acid according to any of claims 1 to 6, comprising the following steps:
(a) full chemical synthesis in a manner known per se, or
(b) chemical synthesis of oligonucleotides, labeling the oligonucleotides, hybridizing the oligonucleotides to DNA from a genomic or cDNA library generated from genomic DNA or mRNA from plant cells, selection of positive clones, and isolation of the hybridizing DNA from positive clones, or
(c) chemical synthesis of oligonucleotides and amplification of the target DNA by PCR.

18. Process of preparing a polypeptide according to claim 14, comprising
(a) cultivating of a host cell according to any of claims 11 to 13 under conditions which ensure the expression of the nucleic acid according to any of claims 1 to 6, or
(b) expressing a nucleic acid according to any of claims 1 to 6 in an in-vitro system, and
(b) obtaining the polypeptide from the cell, the culture medium or the in vitro system.

19. Process of finding a chemical compounds which binds to a polypeptide according to claim 14 or 15, comprising the following steps:
(a) contacting a host cell according to any of claims 11 to 13 or a polypeptide according to claim 14 or 15 with a chemical compound or a mixture of chemical compounds under conditions which permit the interaction of a chemical compound with the polypeptide, and
(b) determining the chemical compound which specifically binds to the polypeptide.

20. Process of finding a compound which modifies the expression of polypeptides according to claim 14, comprising the following steps:
(a) contacting a host cell according to any of claims 11 to 13 with a chemical compound or a mixture of chemical compounds,
(b) determining the polypeptide concentration, and
(c) determining the compound which specifically affects the expression of the polypeptide.

21. Use of a nucleic acid according to any of claims 1 to 6, of a DNA construct according to claim 8, of a vector according to claim 9 or 10, of a host cell according to any of claims 11 to 13, of a polypeptide according to claim 14 or 15 or of an antibody according to claim 16 for finding novel herbicidally active compounds.

22. Use of a modulator of a polypeptide according to claim 14 or 15 as plant growth regulator or herbicide.

23. Use of a nucleic acid according to any of claims 1 to 6, of a DNA construct according to claim 8, a vector according to claim 9 for the generation of transgenic plants.

24. Transgenic plant, plant part, protoplast, plant tissue or plant propagation material, characterized in that, following the introduction of a nucleic acid according to any of claims 1 to 6, a DNA construct according to claim 8 or a vector according to claim 9, the intracellular concentration of a polypeptide according to claim 14 is increased or reduced in comparison with the corresponding wild-type cells.

25. Plant, plant part, protoplast, plant tissue or plant propagation material, characterized in that it comprises a polypeptide according to claim 15 whose biological activity or expression pattern is modified in comparison with the corresponding endogenous polypeptides.

26. Process of generating plants, plant parts, protoplasts, plant tissues or plant propagation materials according to claim 25, characterized in that a nucleic acid according to any of claims 1 to 6 or a regulatory region according to claim 7 is modified by endogenous mutagenesis.


SEQUENCE LISTING (2 sequences)

SEQ ID NO: 1 - DNA, 2672 bp, Nicotiana tabacum
  CDS (166)..(2547)
tctaagccgcgttttcctttactttgattcttcaacacacccaaatcataatcctctccctcttaaagctgcttcaaacctcacaatattatacaaatctttgaaaataattccttcttttattctctaaaacaccatgaaagcattttccatcacaccatttctatggccattcttgataaaaaaactcaccttttcttttctttagtacttctttgctttctcatttccctttcacccatttcttcactttcaactgttgccatttcaaaaacttctaaccaaacactaatttgtgcattgatttcctcctcctcatttcctcaacaatcttctctcaattgtactagttttcctgaaggaattcaaatccctttgaatccttcagtttacttttctggaattgtaggtgggaatggtttcctttgtgggttgacttcatcttactcttcttctacttcaatcatggtgtgttggagattcttaaacaatggtaccaacttgtcttacaaaagtatttatcttggtccattgatcacaaatcttgattccggtaattcccacatttgtggaattgttaatggaaccaataataggcttgaatgttggcagtggcatgaatttaattcatcaaacagaagtttgatgacttcaaatcttgccgttggagaagattttgtttgtggtttgttgacatttggtcaaatccaatgtttaggaagctttagaaatgtcactgatgctattccttcagggaattacagtgaaattgcatctggttcacaacatgtttgtgctatttccaagaataatagtttggtttgttggggaaatatggtaggagaaaagcctattggccaattcaaatcacttgctttaggtgataataggagttgtgctttgaggattaatgggaaagttgtttgttggggagaaactggttttagtctgccttcatctttgagtggggaattttttgaaacattggaagcaaaacaagacattttctgtggtattgtgacctcaaattattcattgttttgttggggcaatgacattttcaattcaaatccagcagtttttaatggtgtaggagtagttccgggaccatgtactacttcatgtccttgtgtacctttacctaattatgagtcattttgtggtcggggactaatgatatgtcaacattgtgttgggcaagattccagtgtgaatccaccaatcgttaacgggtcgggtccttcactaccaccattgcccccacaaccaatgccatcgccaacgccatctcaaacgagtggaagaagcgatccatggagtaggaggaatgtggcatttctagtggtaggttgtgttggatccttaatgatgttgagtgtccttgttatcttgtttttcaagtattgcaagatcagaggatgcagagtacacgactctggccgccttgatgaggcggggtcaccgccccagcaaggcagccagacgtctcgagttcaagatcaacaaggtactcctcagcccccagtcttggaaaaaagacttagtcaattgattagtataggaaatgggggtcatttagatgaattttcattgcaagtgttacttcaagtgactaataatttctccgacgagcacaaaattgggagtggaagttttggagctgtgtatcatgctacattagaagatgggcgcgaagtagccataaaaagagcagaagcttcagcctcatcttcctatgctggtggcacaaaatatagacaagaggacaaagacaatgcattcctcaatgagctagagtttttgtcgcgcctcaatcacaaaaaccttgttaagctattagggtattgtgaagataacaatgaacgtgtcttgattttcgaatacatgaacaatggcactctccatgaccatctccacgggctcgaaagctcaccactaatgtcatgggttggtaggatcaaggtggcattggacgcggcacgtggcatcgagtacttgcatgagtacgcggtgccaactgtcatccaccgtgacatcaagtcgtccaacatattgcttgatgtcacgtggaatgccaaggtgtccgactttggattgtccttaatgggacctcaggatgacgaaacacacctttctatgcgcgctgctggcacggtaggttacatggaccccgagtactacagactgcaacaactaacgacgaaaagtgatgtgtatagtttcggagtaatgttactagagttgttgtcgggttacaaggcaattcacaagaatgagaataaggtaccaagaaatgtggttgattttgttgtgccatacatagtgcaagatgagattcatagggtattggatcgtagagttccaccaccaacaccttttgaaattgagtctgtggcatatgtaggttatctagcagcagattgtaccacattagaaggtagagatcgtccaactatgactcaagttgtaaatacgctagaaagagccttaaaggcatgtttggctactccaattttctctcggtctaacacggatgattcgtccacataagcagcatgctttgacacatgtatattgtaattcacatttttcttattttcctatatatatttaaattatttttccactcatcgcaaaaaaaaaaaaaaaagtcgacatcgatacgcgtggtc

SEQ ID NO: 2 - protein, 794 aa, Nicotiana tabacum
MAILDKKTHLFFSLVLLCFLISLSPISSLSTVAISKTSNQTLICALISSSSFPQQSSLNCTSFPEGIQIPLNPSVYFSGIVGGNGFLCGLTSSYSSSTSIMVCWRFLNNGTNLSYKSIYLGPLITNLDSGNSHICGIVNGTNNRLECWQWHEFNSSNRSLMTSNLAVGEDFVCGLLTFGQIQCLGSFRNVTDAIPSGNYSEIASGSQHVCAISKNNSLVCWGNMVGEKPIGQFKSLALGDNRSCALRINGKVVCWGETGFSLPSSLSGEFFETLEAKQDIFCGIVTSNYSLFCWGNDIFNSNPAVFNGVGVVPGPCTTSCPCVPLPNYESFCGRGLMICQHCVGQDSSVNPPIVNGSGPSLPPLPPQPMPSPTPSQTSGRSDPWSRRNVAFLVVGCVGSLMMLSVLVILFFKYCKIRGCRVHDSGRLDEAGSPPQQGSQTSRVQDQQGTPQPPVLEKRLSQLISIGNGGHLDEFSLQVLLQVTNNFSDEHKIGSGSFGAVYHATLEDGREVAIKRAEASASSSYAGGTKYRQEDKDNAFLNELEFLSRLNHKNLVKLLGYCEDNNERVLIFEYMNNGTLHDHLHGLESSPLMSWVGRIKVALDAARGIEYLHEYAVPTVIHRDIKSSNILLDVTWNAKVSDFGLSLMGPQDDETHLSMRAAGTVGYMDPEYYRLQQLTTKSDVYSFGVMLLELLSGYKAIHKNENKVPRNVVDFVVPYIVQDEIHRVLDRRVPPPTPFEIESVAYVGYLAADCTTLEGRDRPTMTQVVNTLERALKACLATPIFSRSNTDDSST